(19) United States
(12) Patent Application Publication
(10) Pub. No.: US 2021/0403519 A1
(43) Pub. Date: Dec. 30, 2021

(54) METHODS AND COMPOSITIONS TO INDUCE OR SUPPRESS IMMUNE RESPONSES THROUGH THE USE OF MEMBRANE BOUND COMPLEMENT SPLIT PRODUCTS

(71) Applicant: INSIDEOUTBIO, INC., Charlestown, MA (US)

(72) Inventor: Alan Gordon Herbert, Charlestown, MA (US)

(21) Appl. No.: 17/290,021

(22) PCT Filed: Oct. 25, 2019
(86) PCT No.: PCT/US2019/057993

§ 371 Date: Apr. 29, 2021

Related U.S. Application Data
(60) Provisional application No. 62/754,911, filed on Nov. 2, 2018.
(60) Provisional application No. 62/829,912, filed on Apr. 5, 2019.

(51) Int. Cl. C07K 14/47; A61K 38/17; A61K 38/46; C12N 15/90; C12N 9/22; C12N 15/11; A61K 45/06

(52) U.S. Cl. CPC C07K 14/472; A61K 38/1725; A61K 38/465; C12N 15/907; C07K 2319/02; C12N 15/11; A61K 45/06; C12N 2310/20; C12N 2800/80; C12N 9/22

(57) ABSTRACT

Methods and compositions for stimulating or inhibiting antigen-specific immune responses using surface-anchored complement split products are described heron.

### RELATED APPLICATIONS

This application claims the benefit under 35 USC 119(e) of U.S. Provisional Application No. 62/754,911, filed on Nov. 2, 2018, and U.S. Provisional Application No. 62/829,912, filed on Apr. 5, 2019, both of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Oct. 18, 2019, is named 0371_0005W01_SL.txt and is 50,255 bytes in size.

### BACKGROUND OF THE INVENTION

Various methods have been developed to enhance or suppress the immune response to antigens, including inoculation with bacterial infections; viruses; vaccines prepared from tumor cells; immune-stimulants or adjuvants; immune-modulators that boost the immune response and inhibitors of metabolic enzymes that suppress the immune response The rationale for stimulating the immune response is to provide protection against pathogens and tumors by creating a specific immune response against antigens unique to the disease agent. The rationale for stopping an immune response is to prevent autoimmune diseases that attack self-antigens. Despite progress, there remains unmet needs in either stimulating an antigen-specific response against tumors and pathogens, or in limiting an immune response against self-antigens.

### SUMMARY OF THE INVENTION

Complement proteins, complement protein receptors and proteolytic enzymes associated with the complement activation pathway have been investigated for their roles in the regulation of immunity. Complement can be activated by a number of processes, some external to the cell and some inside the cell [1, 2]. After complement activation a series of complement split products (CSP) are produced by proteolysis of C3 according to the following steps.—

C3→C3a+C3b [1]

C3b→iC3b+C3f [2]

iC3b→C3c+C3dg [3]

C3dg→C3d [4]

C4 also is proteolysed to form CSPs by the following steps[3]

C4→C4a+C4b [5]

C4→iC4b+C4c+C4d [6]

C3d, C3dg and C4d activate immune responses (actCSP), while iC3b and C3c are inhibitory (inhCSP). Both C3, and C4 attach to cell membranes through their thioester domain, forming a covalent linkage with carbohydrates that decorate cell membrane proteins [3]. Specific receptors exist for each class of CSP that regulate immune responses [1, 4, 5]. C3 CSP products play a role in T-cell-based immunity [6] while C4d regulates B-Cell mediated immunity [7, 8].

The use of peptides made by fusing multiple copies of C3d to produce a single protein have previously been described to increase the immunogenicity of antigens in some cases [9-11] but not others [12]. Similar fusion constructs have been expressed in melanoma cells, which are then killed and used to produce a vaccine [13, 14]. C4d modulates Th2 responses that are essential to humoral immunity [7].

Increasing the activity of actCSP of C3d or C3dg or C4d in cells provides a basis for greatly enhancing the immunogenicity of tumors, vaccines and the activity of in vitro engineered antigen-specific immune cells.

Increasing the activity of inhCSP like C3c produces tolerance by binding to different receptors than actCSPs like C3d. For example, C3c binds to the product of the VSIG4 gene that produces the receptor CRIg [5]. CRIg inhibits the activation of T-Cells and macrophages [15, 16]. Deletion of VSIG4 in mice produces autoimmunity associated with inflammatory macrophage activation [16, 17].

The present invention encompasses methods and compositions for the targeted activation of immune cells by actCSPs like C3d, C3dg and C4d to stimulate a cellular or humoral immune response against tumors or pathogens. The invention further comprises methods to activate, or enhance, the immune response by localizing actCSPs to the surface of an antigen-bearing cell, be that a tumor cell or a dendritic cell or blood cell such as a B-Cell or Red Blood Cell or platelet or monocyte or myeloid cell, or any other component of the hematopoietic system, using Glycosylphosphatidylinositol (GPI) [18] anchors or to vesicles, such as exosomes (whether natural or artificial, regardless of species) and nanoparticles that also carry an antigen against which an immune response is desired. As described herein, the methods of the present invention will induce an immune response against tumor specific proteins that makes all parts of the tumor susceptible to attack by the immune system both at the site of administration as well as more distant sites throughout the body.

The present invention further comprises methods to activate, or enhance, the immune response by localizing actCSPs using a fusion protein where the actCSP's are anchored by another partner that localizes them to the same surface as the antigen against which an immune response is desired.

The present invention further comprises methods to activate, or enhance, the immune response by depositing actCSP's on the same surface as the antigen against which as immune response is desired, staring with complement C3 or C4 and treating with known enzymes that result in coating the surface with actCSP's. In the case of C3, Step [1] to Step[3] or to Step [4] would be performed. In the case of C4, step [5] and step [6] would be performed.

The present invention also encompasses methods to express GPI-anchored actCSP in immune cells that are engineered in vitro, such as Chimeric Antigen Receptor (CAR) bearing cells (CAR-cells) [19], then administered to patients to act in vivo. In the case of actCSPs, delivery in vitro to antitumor CAR-cell using the methods described here causes an enhancement of tumor killing. In the case of inhCSPs, delivery in vitro to antitumor CAR-cell enhance their ability to suppress antigen-specific auto-immune or allergic responses.

The present invention also encompasses methods to enhance the effectiveness of nucleic acid vaccines [20] by co-expressing expressing GPI-anchored actCSPs along with GPI-anchored peptide antigens in cells.

The present invention encompasses methods to inhibit antigen-specific immune responses, such as those found in autoimmune and allergic diseases by expressing GPI-anchored inhCSPs in a cell or on a surface bearing the antigen.

The present invention uses C4 CSPs to regulate humoral responses and C3 CSPs to regulate cellular immune responses.

The use of GPI-tags is one feature of these inventions. Localization of GPI anchored proteins to the immune synapse formed between an immune cell and a cell expressing the GPI-anchored CSPs and a specific antigen results in engagement of complement receptors on the immune cell and is one mechanism leading to immune cell activation or suppression necessary for the immune cell to perform its effector function. Another mechanism of immune cell regulation involves the transfer of the GPI anchored CSP along with other surface molecules to the membrane of the immune cell, where interactions with other cells either enhance (stimulatory) or limit (inhibitory) clonal expansion of the CSP coated immune cells. One example would be the transfer of antigen and CSP to a dendritic cell in a lymph node [21].

In the case of actCSPs, immature cytotoxic precursors are licensed to kill cancer cells in an antigen-specific fashion. In the case of T-Cells, the specificity of killing is determined by the antigen receptor(s) on the T-Cell. In the case of tumor cells, killing is specific for cells bearing tumor-antigens recognized by the antigen receptor(s) of the T-Cell.

In the case of inhCSPs, these interactions promote the maturation and survival of immature regulatory cells that suppress immune cell responses against antigens involved in initiating or exacerbating disease.

In the case of CAR-cells and other in vitro engineered immune cells, expression of CSPs by CAR-cells enhances their antigen-specific function when introduced in vivo. The CSPs do not change the targeting of the immune cell by its antigen-specific receptors. Only cells that bear antigens capable of triggering the effector function of the CAR-bearing cell will be targeted. Killing of normal cells non-specifically is avoided.

In the case in vitro engineered antigen-presenting cells, such as dendritic cells or other myeloid cells, expression of CSPs on their surface enhances their antigen-specific function when introduced in vivo, allowing modulation of the immune response against the antigen.

In the case of in vitro engineered particles, such as exosomes and nanoparticles, expression of CSPs on their surface directs them to cells in vivo that modulate the immune response to any antigen also present on or in the particle.

Methods described herein include the construction of a fusion protein containing a CSP sequence fused to a GPI tag that localizes the CSP to the surface of a cell or a particle that also contains or expresses the targeted antigen.

Methods described herein include the construction of a fusion protein containing a protein sequence that localizes the CSP to the surface of a cell or a particle that also contains or expresses the targeted antigen. In contrast to previously described methods [9], the antigen is not fused to the CSP As described herein, the GPI tag can be substituted, or replaced, with a ligand-binding site (referred to herein as “LBS”). Specific LBSs are described in Example 4. In contrast to previous methods, it does not involve an antibody targeting C3d [22-24].

In one embodiment, the fusion protein is expressed from a polynucleotide composed of either DNA or RNA introduced to the target cell that contains sequences necessary for the cellular machinery to add the GPI tag and export it to the cell surface membrane. The expression of the fusion protein may be limited to a particular cell type by use of appropriate promoters and enhancers known to one skilled in the art. The agent may be delivered to the target cell using known delivery vehicles, including without limitation, viral vectors, nanoparticles, liposomes or exosomes that may or may not contained ligands for the target cell on their surface. If the delivery of the construct is via a viral vector, the viral vector can comprise any suitable replicating or non-replicating viral vector for targeting and delivery of the construct into a cell and can be for example, adenovirus, adeno-associated virus or lentivirus. Alternatively, delivery may be localized to the target tissue by injection, electroporation or other mechanical or electrophysiological mechanisms.

Another embodiment of the present invention is the directed/targeted delivery of a premade variant CSP with a GPI tag to the surface of the target cell using known delivery vehicles, including without limitation, viral vectors, nanoparticles, liposomes or exosomes that may or may not contained ligands for the target cell on their surface.

The methods of the present invention comprises an expression vector that targets a cell, wherein the vector comprises a nucleic acid construct that expresses a GPI-anchored CSP or a biologically active variant thereof, or encodes a protein that activates expression of CSP and directs addition of a GPI-anchored tag to the CSP, in the target cell. As a result of contacting a target cell, the immunogenicity of the cell is enhanced by actCSP and the tumor cell becomes more susceptible to attack by the immune system. On the other hand, when inhCSPs are delivered to a cell, the stimulation of negative regulatory immune cells is enhanced, leading to a suppression or inhibition of immune responses.

The methods of the present invention comprises an expression vector that targets a cell, wherein the vector comprises a nucleic acid construct that is expressed and that fuses the CSP to a cell surface, more specifically to a sequence from a cell surface molecule that localizes to an immune synapse, such as, but not limited to those membrane and cytoplasmic sequences involved in the surface location of a Class I or Class 11 Major Histocompatibility (MHC) antigen, a tetraspanin or other membrane proteins that place the CSP on the exterior face of the cell membrane or of an extra-cellular vesicle produced by the cell where the CSP directs (e.g., targeting or focusing) the fusion to the immunological synapse.

The methods of the present invention include CSP's with a mutation that removes the cysteine at the C3 or C4 thio-ester site used by the native molecule to form a covalent bond to molecules on a cell surface.

The methods of the present invention include CSP's with a mutation to the CSP known from the study of human genetic disorders to promote inflammatory disease [25, 26] in the case of actCSP and to produce loss of complement function [27, 28] in the case of inhCSP.

The present invention also covers the delivery of a defined antigen to the target cell along with the CSP. In the case of actCSPs, codelivery with a defined antigen increases immune response to that antigen so as to constitute a vaccine against tumors or pathogens that bear the specified antigen. In the case of inhCSPs, codelivery with a defined antigen decreases or suppresses immune responses associated with allergy and autoimmunity triggered by the specified antigen. The defined antigen may be delivered in a number of ways as known to those experienced in the art. The method of the present invention describes delivery of a defined antigen with a GPI-tag that localizes the agent to the cell surface along with the GPI-anchored CSP to induce an immune response against the antigen in the case of actCSPs or to inhibit or suppress it in the case of inhCSPs.

In a particular embodiment, the subject in the methods of this invention is a mammal, and more particularly, the mammal is a human and can activate immunity using GPI-anchored actCSPs or inhibit it using GPI-anchored inhCSPs.

A particular embodiment of the present invention encompasses methods of treating cancer in an individual, preventing metastasis of the cancer and protecting against reoccurrence of the cancer wherein administering to the individual a therapeutically effective amount of the agent increases the expression of actCSPs in the tumor cells or in the tumor micro-environment.

Another embodiment of the present invention is to create a vaccine against tumors that express a defined antigen so as to provoke an immune response to protect an individual against that tumor type, including applications where the vaccine is delivered locally, to lymph nodes, to other tissues or systemically by injection.

Another embodiment of the present invention is to create a vaccine against a pathogen that expresses a defined antigen so as to provoke an immune response to protect an individual against that pathogen.

The methods described herein using actCSP can be used to treat many different forms of cancers. For example, the cancer can be ovarian, breast, colon or lung cancer.

The method of treating cancer can further encompass administering the actCSP agents concurrently with, or sequentially before or after, or in conjunction with, at least one, or more additional or complementary cancer treatments suitable for the treatment of the specific cancer. For example, without limitation, the complementary cancer treatment can be selected from a therapy comprising checkpoint inhibitor; a proteasome inhibitor; immunotherapeutic agent; radiation therapy or chemotherapy. Other suitable additional or complementary cancer therapies are known to those of skill in the art.

Also encompassed by the present invention is a pharmaceutical composition, or compositions, comprising a therapeutically effective amount of the actCSP agents as described herein. The composition additionally can include a pharmaceutically acceptable medium, suitable as a carrier for the agent. The compositions can also include targeting agents to deliver the compositions to specific tumor sites.

The above and other features of the invention including various novel details of construction and combinations of parts, and other advantages, will now be more particularly described with reference to the accompanying drawings and pointed out in the claims. It will be understood that the particular methods and compositions embodying the invention are shown in the drawings and examples by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. The patent or application file contains at least one drawing executed in color. Copies of this patent or application publication with color drawings will be provided by the Office upon request and payment of the necessary fee. Of the drawings:

FIG. 1A-B are schematics that depicts the expression of the different roles of CSP in either activation of immune cells or the licensing of immune cells to become functional effectors. In FIG. 1A, Surface one represents an immune cell bearing an antigen receptor and a CSP receptor. Surface two represents a surface with antigen and a CSP that might be a cell, an exosome or a nanoparticle. In the case of actCSP, the interactions lead to activation of an antigen specific immune cell that stimulates an antigen specific immune response, while in the case of inhCSP, the activated immune cell antigen-specific suppresses the antigen-specific response. Other cells or cell-type involved in these processes are not shown.

FIG. 1B. Once activated, immune cells require licensing before they express effector function. At this stage CSP are expressed on surface 1 of the immune cell and the CSP receptor on another cell involved in licensing effector functions of the activated immune cell. The CSPs may be produced by the activated immune cell [2], or transferred there from antigen presenting cells via trogocytosis or through other mechanisms [29, 30] or by processes that activate complement in the extra-cellular milieu [1, 3].

In both A and B, the CSP proteins described in these inventions can be anchored (shown by triangle) to the cell surface by a GPI anchor or by a fusion to another cell-surface molecule. In cases where the cells are coated with CSP ex vivo, the attachment maybe through the thioester domain after activation of complement to produce CSP's using known procedures. The antigen may be produced by the antigen presenting cell (e.g. a tumor) and integral to surface 2 or anchored by another mechanism (shown by rectangle). Alternatively, the antigen may be expressed on the antigen presenting cells by therapeutic delivery of peptides or nucleic acids to that cell.

FIG. 2A-B depicts nucleic acid constructs suitable for use in the described methods. (A) Construct encodes a GPI-anchored Complement Split Fragment (B) Construct encodes a GPI-anchored Antigen.

FIGS. 3A-B are schematics showing the interaction of C3d and Complement Factor H (CFH) on host cells and how mutations to C3d reduces binding to CFH.

FIG. 3A. On host cells, binding of CFH prevents interaction of C3d with host complement receptors that stimulate an immune response.

FIG. 3B. On non-host cells, mutations to C3d reduce binding to CFH on cancer cells and non-host surfaces. Patches of C3d then can bind to host complement receptors and activate an immune response against antigens that co-localize with C3d on the same surface. (Okemefuna et al., 2009).

FIGS. 4A-B show a two-step procedure for using mutant C3d to amplify immune responses.

FIG. 4A: C3d proteins with mutation to the CFH binding site but with intact complement receptor 2 (CR2) and complement receptor 3 (CR3) binding sites [31-33].

FIG. 4B: Mutated C3d is joined to a ligand binding site to make Reagent 1. Reagent 2 has the cognate ligand joined to the antigen of interest. The interaction between Reagent 1 and Reagent 2 allows delivery of the antigen to an antigen presenting cell in order to stimulate an immune response.

FIG. 5 shows the results of an experiment where nude mice were injected subcutaneously in the flank with 5×10⁶ human 786-0 tumor cells on day 0 and injected with 2 micrograms of plasmid expressing human C3d proteins with a CD55 GPI tag complexed with Avalanche® transfection reagent (EZ Biosystems™ injected intratumorally (blue line) or with transfection reagent alone (red line) on day 7 when the tumor volume was 50 mm³.

FIG. 6A-B. FIGS. 6A and B are graphs showing the results of BALB/c female nude mice injected subcutaneously in the flank with 5×106 human 786-0 tumor cells on day 0 and injected with 5 micrograms of plasmid expressing human. C3d variants with the given SEQ ID NO:2, 9 or 10 (as indicated in the legend) were complexed with Avalanche transfection reagent and injected intratumorally on day 10 when the tumor volume was 100 mm3 and then on day 15. Injection of transfection reagent without DNA was used as an additional control. Mice treated with mutant C3d show a reduction in tumor growth as assessed by measurement of tumor volume and remain healthy as shown by body weight.

FIG. 7A-B show Antibody staining of Tumors. Panel A. Immunoperoxidase staining with a human specific anti-CD44 antibody identifies tumor cells. Panel B. A mouse specific anit-CD335 antibody identifies natural killer cells in treated mice infiltrating the tumor providing evidence of a response. In each panel, the top rows are stained with the antibodies, the middle row with an isotype control and the bottom row without any primary antibody to control for the secondary antibody that is conjugated to the staining reagent. The three left-hand columns are different tumors in the same order in A as in B, while the two right-hand columns are human and mouse normal tissue controls.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

The publications discussed throughout the text are provided solely for their disclosure prior to the filing date of the present application. Nothing herein is to be construed as an admission that the inventors are not entitled to antedate such disclosure by virtue of prior disclosure.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, exemplary methods, and materials are described herein.

General texts, which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”). Examples of protocols sufficient to direct persons of skill through in vitro amplification methods, including the polymerase chain reaction (PCR), the ligase chain reaction (LCR), Q.beta.-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13: 563-564. Improved methods for cloning in vitro amplified nucleic acids are described in Wallace et al., U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated.

The terms “cell”, “exosome” and “extra-cellular vesicle” areused in reference to methods or systems that produce surfaces bearing CSPs with or without antigens and are used without respect to species.

The terms “vector”, “vector construct” and “expression vector” mean the vehicle by which a DNA or RNA sequence (e.g. a foreign gene) can be introduced into a host cell, so as to transform the host and promote expression (e.g. transcription and translation) of the introduced sequence. Vectors typically comprise the DNA of a transmissible agent, into which foreign DNA encoding a protein is inserted by restriction enzyme technology. A common type of vector is a “plasmid”, which generally is a self-contained molecule of double-stranded DNA that can readily accept additional (foreign) DNA and which can readily introduced into a suitable host cell. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g., antibiotic resistance, and one or more expression cassettes.

In one embodiment, the viral vector can be a replication competent retroviral vector capable of infecting only replicating tumor cells with particular mutations [34-36]. In one embodiment, a replication competent retroviral vector comprises an internal ribosomal entry site (IRES) 5′ to the heterologous polynucleotide encoding, e.g., a cytosine deaminase, miRNA, siRNA, cytokine, receptor, antibody or the like. When the heterologous polynucleotide encodes a non-translated RNA such as siRNA, miRNA or RNAi then no IRES is necessary, but may be included for another translated gene, and any kind of retrovirus (see below) can be used. In one embodiment, the polynucleotide is 3′ to an ENV polynucleotide of a retroviral vector. In one embodiment the viral vector is a retroviral vector capable of infecting targeted tumor cells multiple times (5 or more per diploid cell).

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter.

The terms “gene editing” or “gene editing techniques” as described herein can include RNA-mediated interference (referred to herein as RNAi, or interfering RNA molecules), or Short Hairpin RNA (shRNA) or CRISPR-Cas9 and TALEN [37]. See e.g., Agrawal. N. et al., Microbiol Mol Biol Rev. 2003 December; 67(4): 657-685; Moore, C. B., et al. Methods Mol Biol. 2010; 629: 141-158; Doudna, J. A. and Charpentier, E. Science vo. 346, 28 Nov. 2014; Sander, J. D. and Joung, K. Nature Biotech 32, 347-355 (2014); U.S. Pat. No. 8,697,359; Nemudryo, A. A. ACTA Naturae vol. 6, No. 3(22) 2014. Anti-sense RNA can also be used. (Gleave, M. and Monia, B., Nature Reviews Cancer 5, 468-479 (June 2005)). The term “gene therapy” generally means a method of therapy wherein a desired gene/genetic sequence is inserted into a cell or tissue (along with other sequences necessary for the expression of the specific gene). See, for example, genetherapynet.com for description of gene therapy techniques.

The term “subject” as used herein can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “Glycosylphosphatidylinositol (GPI) anchor” refers to a structure consisting of both a lipid and carbohydrate portion covalently bound to proteins that localizes proteins to cell membranes. It is used in the sense described by Heider at al. [18]

The term “cancer” or “tumor” includes, but is not limited to, solid tumors and blood borne tumors. These terms include diseases of the skin, tissues, organs, bone, cartilage, blood and vessels. These terms further encompasses primary and metastatic cancers. Biomarkers identifying the expression of C3, C3b, iC3b, C3c, C3d, C4, C4d, C5, C3aR1, C5aR1, C5aR2, C1R, C1RL, CR2, C1QBP, CD46, CD55, CD59, or LAIR1 in tumors provide one means of selecting patients for treatment, whether the biomarker is detected by RNA expression, antibody or other reagents that allow quantitation of these molecules.

The term “antigen” is defined as any molecule that a T-Cell or B-Cell receptor has specificity for, or any molecule targeted by Natural Killer Cells or other Innate Cells that specifically targets their effector function such as cytotoxic killing of cells, release of growth factors, lymphokines or cytokines. (Microbiology and Immunology On-line, Edited by Richard Hunt, PhD; www.microbiologybook.org/mayer/antigens2000.htm)

The term “CAR” refers to any chimeric antigen receptor introduced into immune cells for therapeutic purposes [38].

The term “CSP” refers to complement split products produced by proteolysis of complement proteins [6]. For the purposes of this invention, it specifically refers to proteolytic fragments produced from complement components C3 and C4 that activate immune responses (“actCSP”) or inhibit them (“inhCSP”).

The methods and compositions of the present invention may be used to treat any type cancerous tumor or cancer cells. Such tumors/cancers may be located anywhere in the body, including without limitation in a tissue selected from brain, colon, urogenital, lung, renal, prostate, pancreas, liver, esophagus, stomach, hematopoietic, breast, thymus, testis, ovarian, skin, bone marrow and/or uterine tissue. Cancers that may treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

A “therapeutically effective” amount as used herein refers to an amount sufficient to have the desired biological effect (for example, an amount sufficient to express the GPI-anchored CSPs to prducethe desired effect on the underlying disease state (for example, an amount sufficient to inhibit tumor growth in a subject, produce an immune response to an antigen or to inhibit autoimmune disease) in at least a sub-population of cells in a subject at a reasonable benefit/risk ratio applicable to any medical treatment. Determination of therapeutically effective amounts of the agents used in this invention, can be readily made by one skilled in the art, by the use of known techniques and by observing results obtained under analogous circumstances. The amounts/dosages may be varied depending upon the requirements of the subject in the judgment of the treating clinician; the severity of the condition being treated and the particular composition being employed. In determining the therapeutically effective amount, a number of factors are considered by the treating clinician, including, but not limited to: the specific disease state; pharmacodynamic characteristics of the particular agent and its mode and route of administration; the desired time course of treatment; the species being treated; its size, age, and general health; the specific disease involved; the degree of or involvement or the severity of the disease; the response of the individual patient; the particular agent administered; the mode of administration; the bioavailability characteristics of the preparation administered; the dose regimen selected; the kind of concurrent treatment (i.e., the interaction of the agent with other co-administered agents); and other relevant circumstances.

For example, as described herein, the amino acid sequence of the iC3b, C3c, C3d, C4b and C4d proteins can be truncated/mutated/altered to produce biologically active peptides or variants. Such peptides derived from the iC3b, C3c, C3d, C4b and C4d protein can be synthesized, or otherwise produced and evaluated for their biological activity. Biological activity can include binding of iC3b, C3c, C3d, C4b and C4d peptides to MHC, or change of sites of proteolysis by proteases such as metalloproteinases, or include sites of proteolysis that result in removal of the GPI tag so that it is released into the extracellular environment. Mutations can specifically increase MHC binding to increase immunomodulation.

In certain embodiments, the agents described for use in this invention can be combined with other pharmacologically active compounds (“additional active agents”) or peptide antigens (“antigens”) known in the art according to the methods and compositions provided herein. Additional active agents can be large molecules (e.g., proteins, lipids, carbohydrates) or small molecules (e.g., synthetic inorganic, organometallic, or organic molecules). In one embodiment, additional active agents independently or synergistically help to treat cancer.

For example, certain additional active agents are anti-cancer chemotherapeutic agents. The term chemotherapeutic agent includes, without limitation, platinum-based agents, such as carboplatin and cisplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon alfa, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine or agents targeted at specific mutations within tumor cells.

Further, the following drugs may also be used in combination with an antineoplastic agent, even if not considered antineoplastic agents themselves: dactinomycin; daunorubicin HCl; docetaxel; doxorubicin HCl; epoetin alfa; etoposide (VP-16); ganciclovir sodium; gentamicin sulfate; interferon alfa; leuprolide acetate; meperidine HCl; methadone HCl; ranitidine HCl; vinblastin sulfate; and zidovudine (AZT). For example, fluorouracil has recently been formulated in conjunction with epinephrine and bovine collagen to form a particularly effective combination.

Still further, the following listing of amino acids, peptides, polypeptides, proteins, polysaccharides, and other large molecules may also be used in conjunction with the invention: checkpoint inhibitors that target for example, PD-1 and CTLA-4, interleukins 1 through 37, including mutants and analogues; interferons or cytokines, such as interferons .alpha., .beta., and .gamma.; hormones, such as luteinizing hormone releasing hormone (LHRH) and analogues and, gonadotropin releasing hormone (GnRH); growth factors, such as transforming growth factor-.beta. (TGF-.beta.), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-.alpha. & .beta. (TNF-.alpha. & .beta.); invasion inhibiting factor-2 (IIF-2); bone morphogenetic proteins 1-7 (BMP 1-7); somatostatin; thymosin-.alpha.-1; .gamma.-globulin, superoxide dismutase (SOD); complement factors; anti-angiogenesis factors; antigenic materials; and pro-drugs.

Chemotherapeutic agents for use with the compositions and methods of treatment described herein include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil, bisantrene; edatraxate: defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

The compositions and methods of the invention can comprise or include the use of other biologically active substances, including therapeutic drugs or pro-drugs, for example, other chemotherapeutic agents or antigens useful for cancer vaccine applications. Various forms of the chemotherapeutic agents and/or additional active agents may be used. These include, without limitation, such forms as uncharged molecules, molecular complexes, salts, ethers, esters, amides, and the like, which are biologically active.

The agents and substances described herein can be delivered to the subject in a pharmaceutically suitable, or acceptable or biologically compatible carrier. The terms “pharmaceutically suitable/acceptable” or “biologically compatible” mean suitable for pharmaceutical use (for example, sufficient safety margin and if appropriate, sufficient efficacy for the stated purpose), particularly as used in the compositions and methods of this invention.

The compositions described herein may be delivered by any suitable route of administration for treating the cancer, including orally, nasally, transmucosally, ocularly, rectally, intravaginally, parenterally, including intramuscular, subcutaneous, intramedullary injections, as well as intrathecal, direct intraventricular, intravenous, intra-articular, intra-sternal, intra-synovial, intra-hepatic, through an inhalation spray, or other modes of delivery known in the art.

The nucleic acid sequence for C3, including the CSPs iC3b, C3d and C3dg can be found e.g., in Proc. Natl. Acad. Sci. USA, vol. 82, pp. 708-712, February 1985). The term “C3d” as used herein is intended to encompass both C3d and C3dg and the term “iC3b” is used to encompass “C3c”. The nucleic acid sequence for the C3aR can be found at “C3AR1 complement C3a receptor 1 [Homo sapiens (human)]” Gene ID: 719, www.ncbi.nlm.nih.gov/gene, updated on 6 Aug. 2017. The nucleic acid sequence for the C5a receptor can be found at “C5AR1 complement C5a receptor 1 [Homo sapiens (human)]” Gene ID: 728, www.ncbi.nlm.nih.gov/gene, updated on 29 Aug. 2017. C1R complement C1r [Homo sapiens (human)], Gene ID: 715, www.ncbi.nlm.nih.gov/gene, updated on 3 Sep. 2017, C1RL complement C1r subcomponent like [Homo sapiens (human), Gene ID: 51279, www.ncbi.nlm.nih.gov/gene, updated on 3 Sep. 2017, C5AR2 complement component 5a receptor 2 [Homo sapiens (human)], Gene ID: 27202, www.ncbi.nlm.nih.gov/gene, updated on 3 Sep. 2017, C1QBP complement C1q binding protein [Homo sapiens (human)], Gene ID: 708, www.ncbi.nlm.nih.gov/gene, updated on 3 Sep. 2017, CR2 complement C3d receptor 2 [Homo sapiens (human)], Gene ID: 1380, www.ncbi.nlm.nih.gov/gene, updated on 3 Sep. 2017, CD46 molecule [Homo sapiens (human)], Gene ID: 4179, www.ncbi.nlm.nih.gov/gene, updated on 3 Sep. 2017, CD55 molecule (Cromer blood group) [Homo sapiens (human)], Gene ID: 1604, www.ncbi.nlm.nih.gov/gene, updated on 6 Sep. 2017, CD59 molecule (CD59 blood group) [Homo sapiens (human)], Gene ID: 966, www.ncbi.nlm.nih.gov/gene, updated on 3 Sep. 2017 and LAIR1 leukocyte associated immunoglobulin like receptor 1 [Homo sapiens (human)], Gene ID: 3903, www.ncbi.nlm.nih.gov/gene, updated on 3 Sep. 2017. The nucleic acid sequence for the proteases cathepsin L [Homo sapiens (human)], CTSL, Gene ID: 1514 and cathepsin S [Homo sapiens (human)], CTSS, Gene ID: 1520, can be found at www.ncbi.nlm.nih.gov/gene, updated on 3 Sep. 2017.

A listing of known C3 and other complement component variants is given at the Leiden Open Variation Database (databases.lovd.nl/shared % genes).

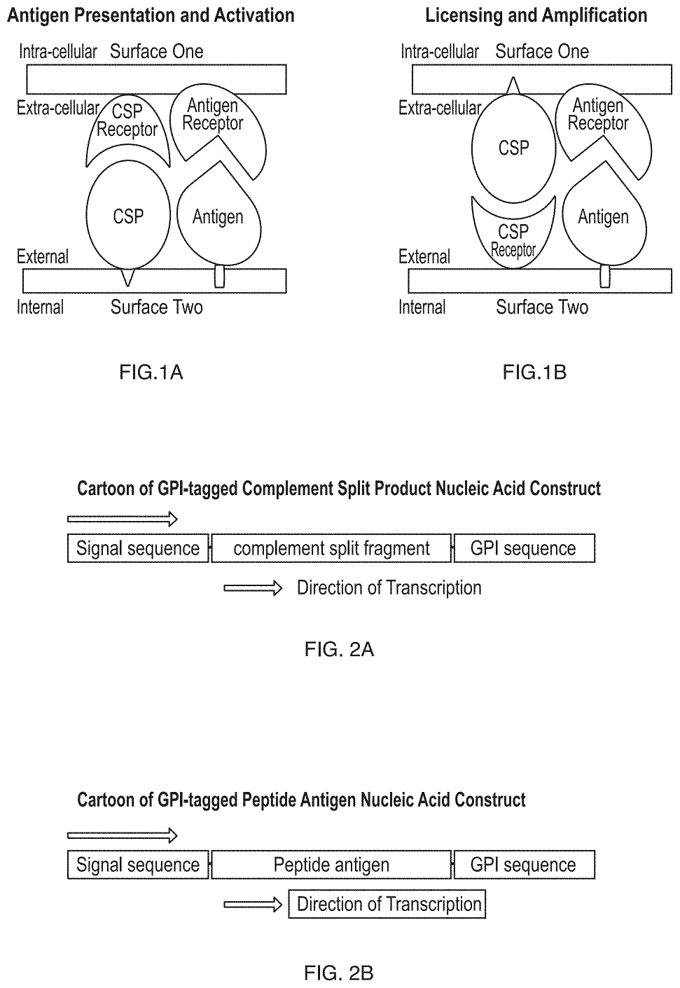

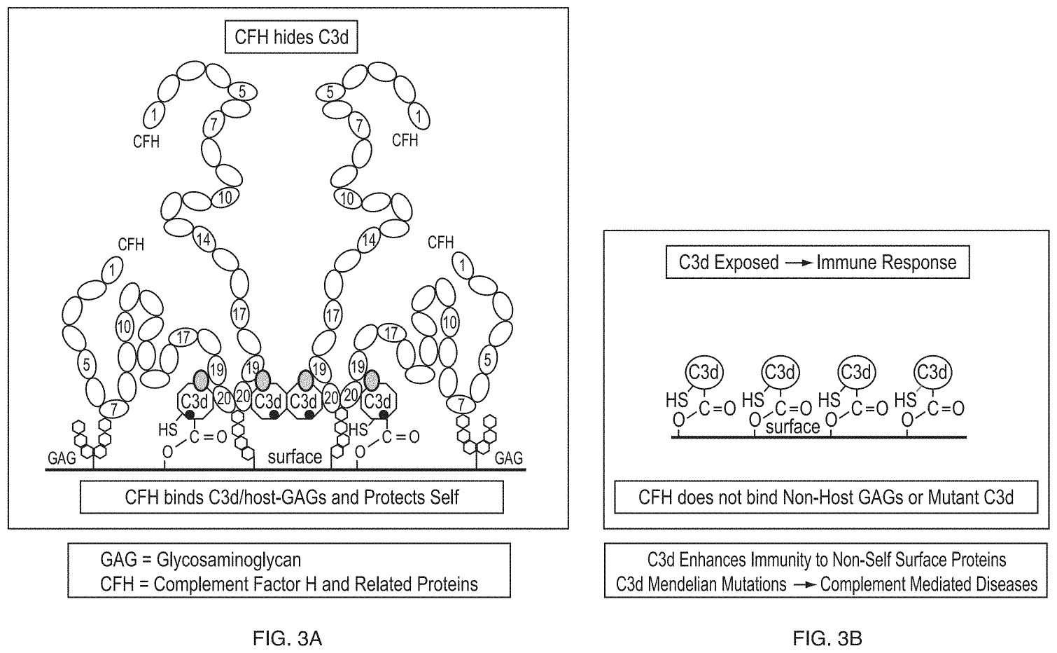

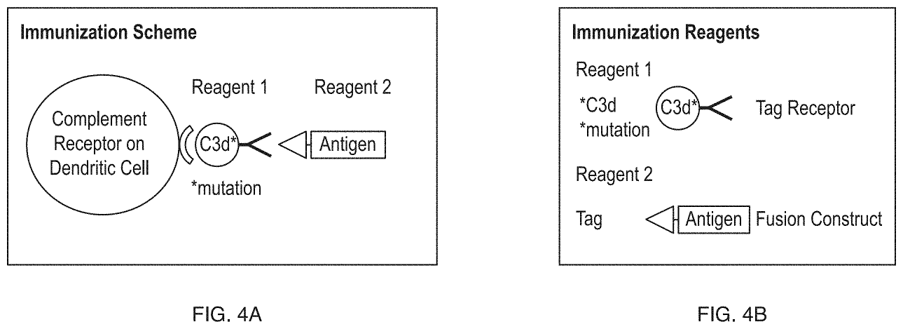

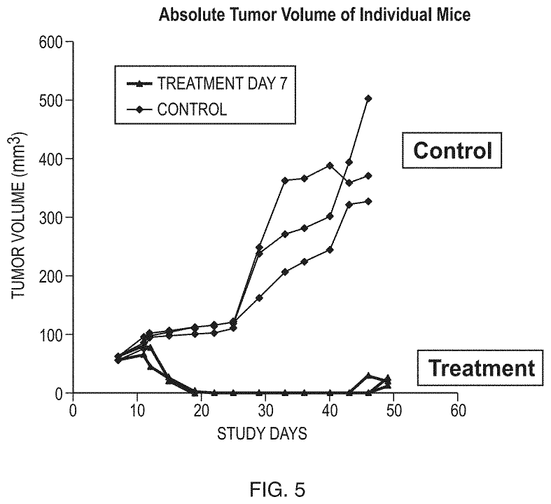

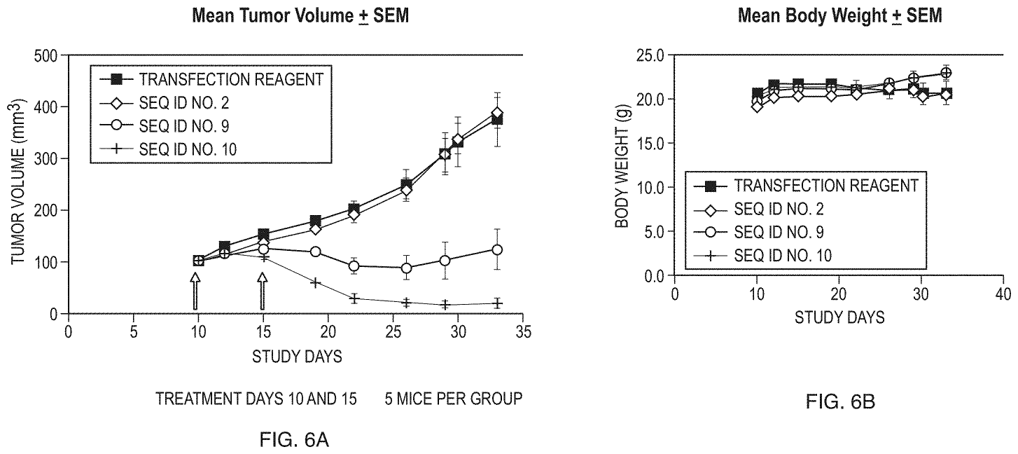

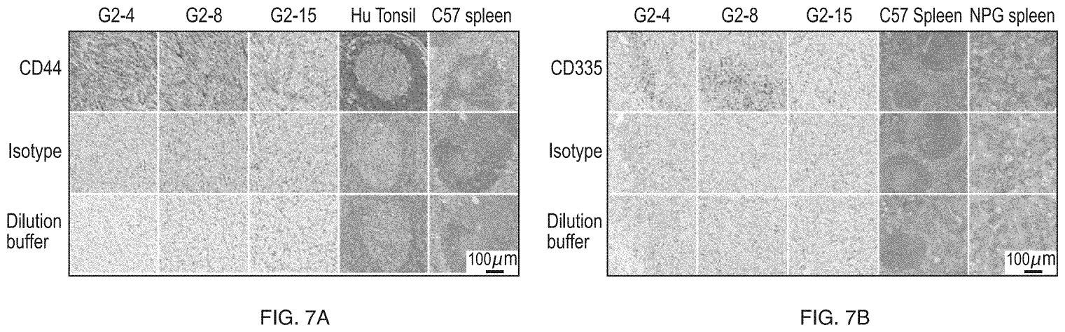

For example, a gene editing technique to produce the C3d or C3c transcript within tumors can be used so that the protein product is targeted to the cell surface membrane as described in this invention (see e.g., U.S. Pat. No. 8,697,359 for a description of CRISPR techniques). Delivery of CRISPR/CAS9 with a sgRNAs to C3 (excluding the C3d sequence) and the nucleic acid sequences for C3d or C3d derived peptides, to a tumor cell can be provided by use of a viral vector. Delivery of CRISPR/CAS9 with a sgRNAs to C3 (excluding the C3c sequence) and the nucleic acid sequences for C3c or C3c derived peptides to a tumor cell along with other sequences necessary for targeting of the CSP transcripts that are introduced into cleavage sites during the process of repair, can be provided by use of a viral vector. A number of viral vectors have been used in humans and these can be used to transduce the genetic material in different cell types. Such methods are known to those of skill in the art. Means to target the vectors for specific delivery of the constructs to the tumor cells of interest are also known to those of skill. For example, genetically engineered vectors exist where the capsid is modified to contain ligands for receptors that facilitate viral entry onto a particular cell type. An example is given in FIG. 1. This construct also includes a reporter gene that allows efficiency of transduction of the virus into the tumor to be quantitated.

The above approaches can be combined with other cancer therapies including immune-modulators such as checkpoint inhibitor ligands for PD-1 CTLA-4, ICOS, OX40; reagents against C3a and C5a receptors; lymphokines, cytokines and their receptors and strategies designed to increase major and minor histocompatibility antigens. Additionally, the methods of the present invention can be combined with other standard cancer therapies such as radiotherapy and chemotherapy.

### EXAMPLES

### Example 1: General Description of Fusion Protein Sequences

The fusion proteins as described herein comprise three parts:
1. A signal sequence directing export to the cell surface membrane
2. The C3d sequence with a mutation at the thioester site of glutamine to alanine at the thioester site of C3d t(he additional constructs described herein may use the wildtype residue at this position)
3. A sequence directing the attachment of the GPI tag

Over 150 proteins are naturally processed to add a GPI anchor and can be used as sources for parts 1 and 3 of the fusion protein [39].

The fusion contains, for example, the following sequences, given as single letter amino acid codes, for each part.

### Example 2: Constructs Containing Mutated C3d

C3d with residues highlighted in red/bold that are mutated to a different amino acid such as alanine, so as to reduce the binding affinity of C3d for CFH and related ligands, while preserving binding sites on C3d for complement receptors CR2 and CR3 [31-33]. The complement receptors take up the mutant C3d along with antigens on the same surface that co-localize with C3d to direct the processing of the antigen along immunostimulatory pathways that provoke an antigen-specific response (FIG. 3).

(C3d Sequence SEQ ID NO: 2)
LDAERLKHLIVTPSGCGEANMIGMTPTVIAVHYLDETEQWEKFGLEKRQ
GALEIKKGYTQQLAFRQPSSAFAAFVKRAPSTWLTAYVVKVFSLAVNLI
AIDSQVLCGAVKWLILEKQKPDGVFQEDAPVIHQEMIGGLRNNNEKDMA
LTAFVLISLQEAKDICEEQVNSLPGSITKAGDFLEANYMNLQRSYTVAI
AGYALAQMGRLKGPLLNKFLTTAKDKNRWEDPGKQLYNVEATSYALLAL
LQLKDFDFVPPVVRWLNEQRYYGGGYGSTQATFMVFQALAQYQKDAPDH
QELNLDVSLQL
Any combination of mutations from SEQ ID NO: 2
above, examples of which are
(SEQ ID NO: 9)
MTVARPSVPAALPLLGELPRLLLLVLLCLPAVWGLDAERLKHLIVTPSG
CGEQNMIGMTPTVIAVHYLDETEQWEKFGLEKRQGALELIKKGYTQQLA
FRQPSSAFAAFVKRAPSTWLTAYVVKVFSLAVNLIAIDSQVLCGAVAWL
ILEKQKPAGVFQEDAPVIHQEMIGGLRNNNEKDMALTAFVLISLQEAKD
ICEEQVNSLPGSITKAGDFLEANYMNLQRSYTVAIAGYALAQMGRLKGP
LLNKFLTTAKDKNRWEDPGKQLYNVEATSYALLALLQLKDFDFVPPVVR
WLNEQRYYGGGYGSTQATFMVFQALAQYQKDAPDHQELNLDVSLQLSGT
TSGTTRLLSGHTCFTLTGLLGTLVTMGLLT
And
(SEQ ID NO: 10)
MTVARPSVPAALPLLGELPRLLLLVLLCLPAVWGLDAERLKHLIVTPSG
CGEQNMIGMTPTVIAVHYLDETEQWEKFGLEKRQGALELIKKGYTQQLA
FRQPSSAFAAFVKRAPSTWLTAYVVKVFSLAVNLIAIDSQVLCGAVKWL
ILEKQKLDGVFQEDAPVIHQEMIGGLRNNNEKDMALTAFVLISLQEAKD
ICEEQVNSLPGSITKAGDFLEANYMNLQRSYTVAIAGYALAQMGRLKGP
LLNKFLTTAKDKNRWEDPGKQLYNVEATSYALLALLQLKDFDFVPPVVR
WLNEQRYYGGGYGSTQATFMVFQALAQYQKDAPDHQELNLDVSLQLSGT
TSGTTRLLSGHTCFTLTGLLGTLVTMGLLT

### Example 3. Constructs Containing Mutated C4d

C4d residues highlighted in red/bold mutated to a different amino acid such as alanine or leucine or any combination of them.

(SEQ ID NO: 11)
TLEIPGNSDPNMIPDGDFNSYVRVTASDPLDTLGSEGALSPGGVASLLR
LPRGCGEATMIYLAPTLAASRYLDKTEQWSTLPPETKDHAVDLIQKGYM
RIQQFRKADGSYAAWLSRDSSTWLTAFVLWLSLAQEQVGGSPEKLQETS
NWLLSQQQADGSFQDPCPVLDRSMQGGLVGNDETVALTAFVTIALHHGL
AVFQDEGAEPLKQRVEASISKANSFLGEKASAGLLGAHAAAITAYALTL
TKAPVDLLGVAHNNLMAMAQETGDNLYWGSVTGSQSNAVSPTPAPRNPS
DPMPQAPALWIETTAYALLHLLLHEGKAEMADQASAWLTRQGSFQGGFR
STQDTVIALDALSAYWIASHTTEERGLNVTLSSTGR

### Example 4: Immunization Reagents/Constructs

Described herein are molecular constructs in which the C3d or C4d variants where the GPI tag that binds specifically to a surface is replaced with a different ligand binding site (LBS) for use with non-surface bound antigens to create an immunization construct (for example, Reagent 1 of FIG. 4) Reagent 1 is specific for a water soluble ligand which is joined to an antigen, called, e.g., Reagent 2, (in FIG. 4) against which the immune response is desired, or which co-localizes on a surface with another molecule against which an immune response is desired

Reagent 1 (FIG. 4) is administered separately from Reagent 2 so that it is pre-bound to the target cell (e.g., the antigen presenting cell). Reagent 2, with an antigen joined to the ligand specific for the LBS of Reagent 1, is administered at a later time point, causing it to be taken up by the antigen-presenting cells coated with Reagent 1 to stimulate an immune response that is specific for the antigen of Reagent 2.

The LBS of Reagent 1 may be an antigen-specific antibody, a lectin specific for abnormal glycoproteins on a cancer cell (for example N-glycolylneuraminic acid by the B subunit of the subtilase cytotoxin) antibodies specific for a virus (for example, antibodies derived from individuals immune to a particular virus), a peptide that binds specifically to theligand, a DNA/RNA aptamer that binds specifically to the target or a nucleic acid, modified or not, that can bind sequence specifically to another nucleic acid.

Joining of the constituents (also referred to herein as “subparts”) to create either Reagent 1 or Reagent 2 can be accomplished by, for example, fusion of gene sequences, the use of chemical crosslinkers or can be implemented using high affinity noncovalent bonds such as those based on the avidin-biotin interaction. Such reagents as described herein can be used for immunizing subjects against an infectious agent or an antigen highly expressed on cancer cells.

### Example 5: A GPI-Anchored C3d Construct (SEQ ID NO: 12)

a.
(SEQ ID NO: 1)
MTVARPSVPA ALPLLGELPR LLLLVLLCLP AVWG (signal
sequence from CD55)
b.
(SEQ ID NO: 2)
LDAERLKHLIVTPSGCGEANMIGMTPTVIAVHYLDETEQWEKFGLEKRQ
GALEIKKGYTQQLAFRQPSSAFAAFVKRAPSTWLTAYVVKVFSLAVNLI
AIDSQVLCGAVKWLILEKQKPDGVFQEDAPVIHQEMIGGLRNNNEKDMA
LTAFVLISLQEAKDICEEQVNSLPGSITKAGDFLEANYMNLQRSYTVAI
AGYALAQMGRLKGPLLNKFLTTAKDKNRWEDPGKQLYNVEATSYALLAL
LQLKDFDFVPPVVRWLNEQRYYGGGYGSTQATFMVFQALAQYQKDAPDH
QELNLDVSLQL (C3d Sequence)
c.
(SEQ ID NO: 3)
ENGGTSLSEK TVLLLVTPFL AAAWSLHP (GPI anchor
from CD59)

### Example 6: A GPI-anchored C3c with Full MG6 Domain Construct (SEQ ID NO: 13)

a.
(SEQ ID NO: 4)
MGPTSGPSLL LLLLTHLPLA LG (signal sequence from
C3)
b.
(SEQ ID NO: 5)
(SESPMYSIITPNILRLESEETMVLEAHDAQGDVPVTVTVHDFPGKKLV
LSSEKTVLTPATNHMGNVTFTIPANREFKSEKGRNKFVTVQATFGTQVV
EKVVLVSLQSGYLFIQTDKTIYTPGSTVLYRIFTVNHKLLPVGRTVMVN
IENPEGIPVKQDSLSSQNQLGVLPLSWDIPELVNMGQWKIRAYYENSPQ
QVFSTEFEVKEYVLPSFEVIVEPTEKFYYIYNEKGLEVTITARFLYGKK
VEGTAFVIFGIQDGEQRISLPESLKRIPIEDGSGEVVLSRKVLLDGVQN
PRAEDLVGKSLYVSATVILHSGSDMVQAERSGIPIVTSPYQIHFTKTPK
YFKPGMPFDLMVFVTNPDGSPAYRVPVAVQGEDTVQSLTQGDGVAKLSI
NTHPSQKPLSITVRTKKQELSEAEQATRTMQALPYSTVGNSNNYLHLSV
LRTELRPGETLNVNFLLRMDRAHEAKIRYYTYLIMNKGRLLKAGRQVRE
PGQDLVVLPLSITTDFIPSFRLVAYYTLIGASGQREVVADSVWVDVKDS
CVGSLVVKSGQSEDRQPGSGSEFPESWLWNVEDLKEPPKNGISTKLMNI
FLKDSITTWEILAVSMSDKKGICVADPFEVTVMGPGQQMTLKIEGDHGA
RVVLVAVDKGVFLNKKNKLTQSKIWDVVEKADIGCTPGSGKDYAGVFSD
AGLTFTSSSGQQTAQTAELQCPQP (C3c sequence)
c.
(SEQ ID NO: 7)
SGTTSGTTRLLSGHTCFTLTGLLGTLVTMGLLT (GPI anchor
from CD55)

### Example 7: A GPI-C3c Fusion with MG6 Domain Residues Only from the C3 β-Chain (SEQ ID NO: 14)

a.
(SEQ ID-NO: 4)
MGPTSGPSLLLLLLTHLPLALG_(signal sequence from C3)
b.
(SEQ ID NO: 6)
SPMYSIITPNILRLESEETMVLEAHDAQGDVPVTVTVHDFPGKKLVLSS
EKTVLTPATNHMGNVTFTIPANREFKSEKGRNKFVTVQATFGTQVVE
KVVLVSLQSGYFIQTDKTIYTPGSTVLYRIFTVNHKLLPVGRTVMVNI
ENPEGIPVKQDSLSSQNQLGVLPLSWDIPELVNMGQWKIRAYYENSPQ
QVFSTEFEVKEYVLPSFEVIVEPTEKFYYIYNEKGLEVTITARFLYGKK
VEGTAFVIFGIQDGEQRISLPESLKRIPIEDGSGEVVLSRKVLLDGVQNP
RAEDLVGKSLYVSATVILHSGSDMVQAERSGIPIVTSPYQIHFTKTPKY
FKPGMPFDLMVFVTNPDGSPAYRVPVAVQGEDTVQSLTQGDGVAKLS
INTHPSQKPLSITVRTKKQELSAEQATRTMQALPYSTVGNSNNYLHLS
VLRTELRPGETLNVNFLLRMDRAHEAKIRYYTYLIMNKGRLLKAGRQ
VREPGQDLVVLPLSITTDFIPSFRLVAYYTLIGASGQREVVADSVWVDV
KDSCVGSLVVKSGQSEDRQPVPGQQMTLKIEGDHGARVVLVAVDKG
VFVLNKKNKLTQSKIWDVVEKADIGCTPGSGKDYAGVFSDAGLTFTS
SSGQQTAQRAELQCPQP (C3c sequence)
c.
(SEQ ID NO: 7)
SGTTSGTTRLLSGHTCFTLTGLLGTLVTMGLLT (GPI anchor
from CD55)

### Example 8: A GPI-Anchored C4a Construct (SEQ ID NO: 15)

This C4d construct comprises a C4d sequence with a mutation of glutamine to alanine at the thioester site of C4d.

a.
(SEQ ID NO: 1)
MTVARPSVPA ALPLLGELPR LLLLVLLCLP AVWG (signal
sequence from CD55).
b.
(SEQ ID NO: 8)
TLEIPGNSDPNMIPDGDFNSYVRVTASDPLDTLGSEGALSPGGVASLLR
LPRGCGEATMIYLAPTLAASRYLDKTEQWSTLPPEKTKDHAVDLIQKGY
MRIQQFRKADGSYAAWLSRDSSTWLTAFVLKVLSLAQEQVGGSPEKL
QETSNWLLSQQQADGSFQDPCPVLDRSMQGGLVGNDETVALTAFVTI
ALHHGLAVFQDEGAEPLKQRVEASISKANSFLGEKASAGLLGAHAAAI
TAYALTLTKAPVDLLGVAHNNLMAMAQETGDNLYWGSVTGSQSNA
WLTRQGSFQGGFRSTQDTVIALDALSAYWIASHTTEERGLNVTLSSTG
R (C4a sequence)
c.
(SEQ ID NO: 7)
SGTTSGTTRLLSGHTCFTLTGLLGTLVTMGLLT (GPI anchor
from CD)

### Example 9: A GPI-Anchored Peptide Antigen Construct

a.
(SEQ ID NO: 1)
MTVARPSVPA ALPLLGELPR LLLLVLLCLP AVWG (signal
sequence from CD55)
b.
Antigenic peptide sequence
c.
(SEQ ID NO: 7)
SGTTSGTTRLLSGHTCFTLTGLLGTLVTMGLLT (GPI anchor
from CD)

References (the references listed in this application are herein incorporated in their entirety by reference):
[1] A. Erdei, N. Sandor, B. Macsik-Valent, S. Lukacsi, M. Kremlitzka, Z. Bajtay, The versatile functions of complement C3-derived ligands, Immunol Rev 274(1) (2016) 127-140.
[2] M. K. Liszewski, M. Kolev, G. Le Friec, M. Leung, P. G. Bertram, A. F. Fara, M. Subias, M. C. Pickering, C. Drouet, S. Meri, T. P. Arstila, P. T. Pekkarinen, M. Ma, A. Cope, T. Reinheckel, S. Rodriguez de Cordoba, B. Afzali, J. P. Atkinson, C. Kemper, Intracellular complement activation sustains T cell homeostasis and mediates effector differentiation, Immunity 39(6) (2013) 1143-57.
[3] S. K. Law, A. W. Dodds, The internal thioester and the covalent binding properties of the complement proteins C3 and C4, Protein Sci 6(2) (1997) 263-74.
[4] G. Bajic, L. Yatime, R. B. Sim, T. Vorup-Jensen, G. R. Andersen, Structural insight on the recognition of surface-bound opsonins by the integrin I domain of complement receptor 3, Proc Natl Acad Sci USA 110(41) (2013) 16426-31.
[5] J. Hofer, F. Forster, D. E. Isenman, M. Wahrmann, J. Leitner, M. A. Holzi, J. J. Kovarik, H. Stockinger, G. A. Bohmig, P. Steinberger, G. J. Zlabinger, Ig-like transcript 4 as a cellular receptor for soluble complement fragment C4d, Faseb J 30(4) (2016) 1492-503.
[6] G. Hajishengallis, E. S. Reis, D. C. Mastellos, D. Ricklin, J. D. Lambris, Novel mechanisms and functions of complement, Nat Immunol 18(12) (2017) 1288-1298.
[7] N. Lu, Y. Li, Z. Zhang, J. Xing, Y. Sun, S. Yao, L. Chen, Human Semaphorin-4A drives Th2 responses by binding to receptor ILT-4, Nat Commun 9(1) (2018) 742.
[8] A. Sahu, J. D. Lambris, Structure and biology of complement protein C3, a connecting link between innate and acquired immunity, Immunol Rev 180 (2001) 35-48.
[9] P. W. Dempsey, M. E. Allison, S. Akkaraju, C. C. Goodnow, D. T. Fearon, C3d of complement as a molecular adjuvant: bridging innate and acquired immunity, Science 271(5247) (1996) 348-50.
[10] A. Segal, Opsonin-enhanced cells, and methods of modulating an immune response to an antigen, 1997, WO1997035619A1.
[11] A. Segal, Vaccine compositions and methods of modulating immune responses, 2002, US20030133942.
[12] Y. Lee, K. M. Haas, D. O. Gor, X. Ding, D. R. Karp, N. S. Greenspan, J. C. Poe, T. F. Tedder, Complement component C3d-antigen complexes can either augment or inhibit B lymphocyte activation and humoral immunity in mice depending on the degree of CD21/CD19 complex engagement, J Immunol 175(12) (2005) 8011-23.
[13] M. I. Cascalho, J. L. Platt, Mutable vaccines, 2002.
[14] J. L. Platt, I. Silva, S. J. Balin, A. R. Lefferts, E. Farkash, T. M. Ross, M. C. Carroll, M. Cascalho, C3d regulates immune checkpoint blockade and enhances antitumor immunity, JCI Insight 2(9) (2017).
[15] L. Vogt, N. Schmitz, M. O. Kurrer, M. Bauer, H. I. Hinton, S. Behnke, D. Gatto, P. Sebbel, R. R. Beerli, I. Sonderegger, M. Kopf, P. Saudan, M. F. Bachmann, VSIG4, a B7 family-related protein, is a negative regulator of T cell activation, J Clin Invest 116(10) (2006) 2817-26.
[16] J. Li, B. Diao, S. Guo, X. Huang, C. Yang, Z. Feng, W. Yan, Q. Ning, L. Zheng, Y. Chen, Y. Wu, VSIG4 inhibits proinflammatory macrophage activation by reprogramming mitochondrial pyruvate metabolism, Nat Commun 8(1) (2017) 1322.
[17] A. G. Small, M. Al-Baghdadi, A. Quach, C. Hii, A. Ferrante, Complement receptor immunoglobulin: a control point in infection and immunity, inflammation and cancer, Swiss Med Wkly 146 (2016) w14301.
[18] S. Heider, J. A. Dangerfield, C. Metzner, Biomedical applications of glycosylphosphatidylinositol-anchored proteins, J Lipid Res 57(10) (2016) 1778-1788.
[19] S. C. Wagner, T. Ichim, Gene editing for immunological destruction of neoplasia, 2017, US20170020922A1.
[20] H. L. Robinson, J. M. Smith, J. Hua, B. Moss, R. R. Amara, L. S. Wyatt, P. L. Earl, T. M. Ross, R. A. Bright, S. T. Butera, Compositions and methods for generating an immune response, 2015, US20150231227A1.
[21] T. G. Phan, I. Grigorova, T. Okada, J. G. Cyster, Subcapsular encounter and complement-dependent transport of immune complexes by lymph node B cells, Nat Immunol 8(9) (2007) 992-1000.
[22] D. T. Fearon, P. W. Dempsey, Modulating the immune response, 2005, CA2207000A1
[23] A. U. Wiestner, M. W. Skarzynski, M. A. Lindorfer, R. P. Taylor, C. Rader, B. Vire, Antibody targeting cell surface deposited complement protein C3d and use thereof, 2016, WO2015105973A1.
[24] V. M. Holers, J. M. Thurman, L. Kulik, S. Tomlinson, Antibodies to the C3d fragment of complement component 3, 2017, US20130129728A1.
[25] E. Bresin, E. Rurali, J. Caprioli, P. Sanchez-Corral, V. Fremeaux-Bacchi, S. Rodriguez de Cordoba, S. Pinto, T. H. Goodship, M. Alberti, D. Ribes, E. Valoti, G. Remuzzi, M. Noris, Combined complement gene mutations in atypical hemolytic uremic syndrome influence clinical phenotype, J Am Soc Nephrol 24(3) (2013) 475-86.
[26] Y. Deng, B. P. Tsao, Updates in Lupus Genetics, Curr Rheumatol Rep 19(11) (2017) 68.
[27] L. Skattum, M. van Deuren, T. van der Poll, L. Truedsson, Complement deficiency states and associated infections, Mol Immunol 48(14) (2011) 1643-55.
[28] G. Sfyroera, D. Ricklin, E. S. Reis, H. Chen, E. L. Wu, Y. N. Kaznessis, K. N. Ekdahl, B. Nilsson, J. D. Lambris, Rare loss-of-function mutation in complement component C3 provides insight into molecular and pathophysiological determinants of complement activity, J Immunol 194(7) (2015) 3305-16.
[29] O. Rechavi, I. Goldstein, Y. Kloog, Intercellular exchange of proteins: the immune cell habit of sharing, FEBS Lett 583(11) (2009) 1792-9.
[30] K. McCoy-Simandle, S. J. Hanna, D. Cox, Exosomes and nanotubes: Control of immune cell communication, Int J Biochem Cell Biol 71 (2016) 44-54.
[31] G. Bajic, S. E. Degn, S. Thiel, G. R. Andersen, Complement activation, regulation, and molecular basis for complement-related diseases, Embo J 34(22) (2015) 2735-57.
[32] T. Kajander, M. J. Lehtinen, S. Hyvarinen, A. Bhattacharjee, E. Leung, D. E. Isenman, S. Meri, A. Goldman, T. S. Jokiranta, Dual interaction of factor H with C3d and glycosaminoglycans in host-nonhost discrimination by complement, Proc Natl Acad Sci USA 108(7) (2011) 2897-902.
[33] J. M. van den Elsen, D. E. Isenman, A crystal structure of the complex between human complement receptor 2 and its ligand C3d, Science 332(6029) (2011) 608-11.
[34] H. Von Melchner, T. Andreu, C. Ebensperger, Self-deleting vectors, 2003, WO2016094888A1.
[35] L. Naldini, T. Dull, D. A. Farson, R. Witt, Method and means for producing high titer, safe, recombinant lentivirus vectors, 2005, WO2016145150A3.
[36] T. Farries, D. Eckland, Cancer therapy, 2017, U.S. Pat. No. 9,603,912B2.
[37] H. Tang, J. B. Shrager, Crispr/cas-mediated genome editing to treat egfr-mutant lung cancer, 2017, US20170145405A1.
[38] C. R. Maldini, G. I. Ellis, J. L. Riley, CAR T cells for infection, autoimmunity and allotransplantation, Nat Rev Immunol 18(10) (2018) 605-616.
[39] T. Kinoshita, M. Fujita, Biosynthesis of GPI-anchored proteins: special emphasis on GPI lipid remodeling, J Lipid Res 57(1) (2016) 6-24.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of enhancing the immunogenicity of an antigen by using actCSP, the method comprising contacting a cell with an agent wherein the agent increases the expression of a GPI-anchored actCSP or a biologically active variant thereof including peptides derived from actCSP in the cell or the cell microenvironment.

2. A method of decreasing the immunogenicity of an antigen by using inhCSP, the method comprising contacting a cell with an agent wherein the agent increases the expression of a GPI-anchored inhCSP or a biologically active variant thereof including peptides derived from inhCSP in the cell or the cell microenvironment.

3. The method of claim 1 wherein the actCSP is C3d or C4d or a peptide incorporating a sequence with greater than 90% similarity to those sequences.

4. The method of claim 2 wherein the inbCSP is iC3b or C3 or a peptide incorporating a sequence with greater than 90% similarity to those sequences.

5. The method of either claim 1 or 2, wherein the first agent comprises a gene-editing agent that increases expression of GPI-anchored CSP on the cell surface.

6. The method of either claim 3 or 4, wherein the gene-editing agent comprises a CRISPR-Cas system construct that increases expression of GPI-anchored CSP on the cell surface.

7. The method of either claim 3 or 4, wherein the gene-editing agent comprises a TALEN construct that increases expression of GPI-anchored CSP on the cell surface.

8. The methods of any one of claims 5 to 7, wherein the gene-editing agent does not decrease or inhibit the expression of GPI-anchored CSP on the cell surface.

9. The method of either claim 1 or 2, wherein the first agent is a nucleic acid construct comprising an RNA encoding a GPI-anchored CSP on the cell surface.

10. The method of claim 1, wherein the first agent is a nucleic acid construct that directs production of a GPI-anchored CSP on the cell surface.

11. The method of any one of claims 5 to 10, wherein the agent is targeted for delivery to surface of a cell using a viral vector, nanoparticle, liposome or exosome.

12. The method of claim 11, wherein the viral vector comprises adenovirus, adeno-associated virus, herpes virus, paramyxovirus or lentivirus.

13. The method of either claim 1 or 2, wherein a second agent comprises an expression vector that increases expression of an antigen to induce an immune response specific for that antigen whereas both the GPI-anchored actCSP and antigen are expressed on the surface of the same cell or to inhibit an immune response whereas both the GPI-anchored inhCSP and antigen are expressed on the surface of the same cell.

14. The method of claim 13, wherein the antigen is tagged by GPI to colocalize it to the same site on the surface of the cell as the GPI-anchored CSP.

15. The method of claim 11, wherein the subject is a mammal.

16. The method of claim 15, wherein the mammal is a human.

17. The method of claim 11, wherein the agent comprises a gene-editing agent that increases the expression of one, or more GPI-anchored CSP and antigens in the same cells.

18. The method of claim 17, wherein the gene-editing agent comprises a CRISPR-Cas system construct that increases the expression of one, or more GPI-anchored CSPs and antigens in the same cells.

19. The method of claim 17, wherein the gene-editing agent comprises a TALEN construct that increases the expression of one, or more GPI-anchored CSPs and antigens in the same cells.

20. The method of claim 17, wherein the gene-editing agent comprises a Meganuclease construct that increases the expression of one, or more GPI-anchored CSPs and antigens in the same cells.

21. The method of claim 17, wherein the gene-editing agent comprises a homologous recombination construct that increases the expression of one, or more GPI-anchored CSPs and antigens in the same cells.

22. The method of claim 17, wherein the gene-editing agent comprises a base editing construct that increases the expression of one, or more GPI-anchored CSPs and antigens in the same cells.

23. The methods of any one of claims 17 to 21, wherein the gene-editing agent does not decrease or inhibit the expression of GPI-anchored CSPs or peptides derived from them in a cell.

24. The method of claim 11, wherein the agent is a nucleic acid construct comprising RNAi, s RNA that increases expression of GPI-anchored CSPs in cells.

25. The method of claim 11, wherein the first agent is a nucleic acid construct that increases expression of GPI-anchored CSPs in cells.

26. The method of any one of claims 17 to 25, wherein the agent is targeted for delivery to the tumor cells using a viral vector, liposome or exosome.

27. The method of claim 26, wherein the viral vector comprises adenovirus, adeno-associated virus, herpes virus, paramyxovirus or lentivirus.

28. The method of claim 13, wherein the second agent comprises an expression vector that targets the cells, wherein the vector comprises a nucleic acid construct that expresses antigens, or a biologically active variant thereof, in the tumor cell.

29. The composition of either claim 24 or 25, wherein the gene-editing agent does not decrease or inhibit the expression of GPI-anchored CSPs in cells or a biologically active variant thereof including peptides derived from CSPs in cells.

30. A method of treating cancer, or preventing metastasis of cancer, the method comprising administering to the subject a therapeutically effective amount of a first agent wherein the first agent increases expression of GPI-anchored actCSPs in cancer cells or other cells that present tumor antigens to the immune system, such as dendritic cells.

31. The method of 30, wherein a therapeutically effective amount of a second agent co-administered wherein the second agent increases the expression of a tumor-expressed antigen in cells or the micro-environment, thereby treating cancer, or preventing metastasis of cancer, or protecting against a reoccurrence of cancer in the subject by inducing an immune response to tumor-specific antigens.

32. The method of claim 30, wherein the first agent comprises a gene-editing agent that decreases or increases expression of GPI-anchored actCSPs in tumor cells.

33. The method of claim 30, wherein the gene-editing agent comprises a CRISPR-Cas system construct that increases expression of GPI-anchored actCSPs in tumor cells.

34. The method of claim 30, wherein the gene-editing agent comprises a TALEN construct that increases expression of GPI-anchored actCSPs in tumor cells.

35. The method of claim 30, wherein the gene-editing agent comprises a Meganuclease construct that increases expression of GPI-anchored actCSPs in tumor cells.

36. The method of claim 30, wherein the gene-editing agent comprises a homologous recombination construct that increases expression of GPI-anchored actCSPs in tumor cells.

37. The method of claim 30, wherein the gene-editing agent comprises a base edting construct that increases expression of GPI-anchored actCSPs in tumor cells.

38. The methods of any one of claims 32 to 37, wherein the gene-editing agent does not decrease or inhibit the expression of actCSPs or a biologically active variant thereof including peptides derived from actCSPs in the cancer cells.

39. The method of claim 30, wherein the first agent is a nucleic acid construct comprising RNA that increases expression of GPI-anchored actCSPs in tumor cells.

40. The method of claim 30, wherein the first agent is a nucleic acid construct that expresses a protein that increases expression of GPI-anchored actCSPs in tumor cells.

41. The method of any one of claims 32 to 40, wherein the agent is targeted for delivery to the cancer cells using a viral vector, liposome or exosome.

42. The method of claim 41, wherein the viral vector comprises adenovirus, adeno-associated virus, herpes virus, paramyxovirus or lentivirus.

43. The method of claim 41, wherein the agent comprises an expression vector that targets the tumor cells, wherein the vector comprises a nucleic acid construct that expresses actCSPs or peptide(s) derived from within, or a biologically active variant thereof, in the cancer cell.

44. The method of either claim 31 or 41, wherein the second agent comprises an expression vector comprises a nucleic acid construct that expresses antigen(s), or a biologically active variant thereof, of a tumor-expressed antigen in the same cell as the first agent.

45. The method of either claim 30 or 31, wherein the first and/or second agent is administered concurrently with, or sequentially before or after at least one other cancer treatment.

46. The method of claim 41, wherein the cancer treatment is administration of a treatment selected from the group consisting of: a checkpoint inhibitor; a proteasome inhibitor; immunotherapy; radiation therapy; chemotherapy.

47. A pharmaceutical composition comprising a therapeutically effective amount of a first agent that increases expression of GPI-anchored actCSPs in tumor cells, or any combination thereof, in a tumor cell, and a therapeutically effective amount of a second agent that increases the expression or activity of a tumor specific antigen or a biologically active variant thereof including peptides derived from the tumor cell or the tumor cell microenvironment, in a pharmaceutically acceptable medium.

48. The composition of claim 47, wherein the first agent comprises a gene-editing agent that increases expression of GPI-anchored actCSPs in tumor cells. within the tumor cell.

49. The composition of claim 47, wherein the gene-editing agent comprises a CRISPR-Cas system construct that increases expression of GPI-anchored actCSPs in tumor cells.

50. The composition of claim 47, wherein the gene-editing agent comprises a TALEN construct that increases expression of GPI-anchored actCSPs in tumor cells.

51. The composition of claim 47, wherein the gene-editing agent comprises a Meganuclease construct that increases expression of GPI-anchored actCSPs in tumor cells.

52. The composition of claim 47, wherein the gene-editing agent comprises a homologous recombination construct that increases expression of GPI-anchored actCSPs in tumor cells.

53. The composition of claim 47, wherein the gene-editing agent comprises a base editing construct that increases expression of GPI-anchored actCSPs in tumor cells.

54. The composition of claim 47, wherein the first agent is a nucleic acid construct comprising RNA that increases expression of GPI-anchored actCSPs in tumor cells.

55. The composition of claim 47, wherein the first agent is a nucleic acid construct that expresses a protein that increases expression of GPI-anchored actCSPs in tumor cells.

56. The composition of any one of claims 48 to 55 wherein the agent is targeted for delivery to the tumor cell using a viral vector, liposome or exosome.

57. The composition of claim 54, wherein the viral vector comprises adenovirus, adeno-associated virus, herpes virus, paramyxovirus or lentivirus.

58. The composition of claim 47, wherein the second agent comprises an expression vector that targets the tumor cells, wherein the vector comprises a nucleic acid construct that expresses antigen or a biologically active variant thereof including peptides in the tumor cells.

59. The composition of claim 46, wherein the first agent comprises a small molecule that increases expression of GPI-anchored actCSPs in tumor cells.

60. The method of either claim 1 or 2, wherein the agent is expressed in a CAR-bearing cell in vitro to be used in vivo to modulate an immune response.

61. A pharmaceutical composition comprising a therapeutically effective amount of an agent that decreases the immunogenicity of an antigen expressed on a cell by using inhCSP, wherein the agent increases the expression of a GPI-anchored inhCSP, or a biologically active variant thereof including peptides derived from inhCSP, or protein fusions including C3c, in the cell or the cell microenvironment in a pharmaceutically acceptable medium.

62. The pharmaceutical composition of claim 61, wherein the inhCSP is iC3b or C3c or a peptide incorporating a sequence with greater than 90% similarity to those sequences.

63. The pharmaceutical composition of 61, wherein the composition comprises a second agent and the second agent comprises an expression vector that increases the expression of an antigen that inhibits an immune response wherein both the GPI-anchored inhCSP and antigen are expressed on the surface of the same cell.

64. A nucleic acid construct comprising a mutated complement split protein.

65. The construct of claim 64 wherein the complement split protein is a C3d protein.

66. The mutated C3d protein of claim 65, comprising a nucleic acid sequence selected form the group consisting of: SEQ ID NO: 2, SEQ ID NO:8 or SEQ ID NO: 9.

67. The construct of claim 64 further comprising a CD 55 signal sequence.

68. The signal sequence of claim 67, wherein the sequence comprises SEQ ID NO: 1.

69. The construct of claim 64 further comprising a GPI anchor sequence.

70. The construct of claim 69 wherein the GPI sequence is a CD59 anchor sequence comprising SEQ ID NO: 3.

71. The construct of claim 69 wherein the GPI sequence is a CD55 anchor sequence comprising SEQ ID NO: 6.

72. The construct of claim 66 further comprising a ligand binding signal.

73. The construct of claim 64 wherein the complement split protein is a C4d protein.

74. The construct of claim 73 wherein the C4d protein comprises SEQ ID NO: 10.

75. The construct of claim 74 further comprising a CD 55 signal sequence.

76. The signal sequence of claim 75, wherein the sequence comprises SEQ ID NO: 1.

77. The construct of claim 74 further comprising a GPI anchor sequence.

78. The construct of claim 77 wherein the GPI sequence is a CD59 anchor sequence comprising SEQ ID NO: 3.

79. The construct of claim 77 wherein the GPI sequence is a CD55 anchor sequence comprising SEQ ID NO: 6.

80. The construct of claim 73 further comprising a ligand binding signal.

81. A method of immunizing a subject against an infectious agent of an antigen expressed on a cancer cell, the method comprising administering to the subject a nucleic acid construct according to any one of claims 64-80.


SEQUENCE LISTING (15 sequences)

SEQ ID NO: 1 - protein, 34 aa, Artificial Sequence
  source: /note=“Description of Artificial Sequence Synthetic polypeptide”
MTVARPSVPAALPLLGELPRLLLLVLLCLPAVWG

SEQ ID NO: 2 - protein, 306 aa, Artificial Sequence
  source: /note=“Description of Artificial Sequence Synthetic polypeptide”
LDAERLKHLIVTPSGCGEANMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQL

SEQ ID NO: 3 - protein, 28 aa, Artificial Sequence
  source: /note=“Description of Artificial Sequence Synthetic peptide”
ENGGTSLSEKTVLLLVTPFLAAAWSLHP

SEQ ID NO: 4 - protein, 22 aa, Artificial Sequence
  source: /note=“Description of Artificial Sequence Synthetic peptide”
MGPTSGPSLLLLLLTHLPLALG

SEQ ID NO: 5 - protein, 708 aa, Artificial Sequence
  source: /note=“Description of Artificial Sequence Synthetic polypeptide”
SPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPGSGSEFPESWLWNVEDLKEPPKNGISTKLMNIFLKDSITTWEILAVSMSDKKGICVADPFEVTVMGPGQQMTLKIEGDHGARVVLVAVDKGVFVLNKKNKLTQSKIWDVVEKADIGCTPGSGKDYAGVFSDAGLTFTSSSGQQTAQRAELQCPQP

SEQ ID NO: 6 - protein, 643 aa, Artificial Sequence
  source: /note=“Description of Artificial Sequence Synthetic polypeptide”
SPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQP

SEQ ID NO: 7 - protein, 33 aa, Artificial Sequence
  source: /note=“Description of Artificial Sequence Synthetic polypeptide”
SGTTSGTTRLLSGHTCFTLTGLLGTLVTMGLLT

SEQ ID NO: 8 - protein, 380 aa, Artificial Sequence
  source: /note=“Description of Artificial Sequence Synthetic polypeptide”
TLEIPGNSDPNMIPDGDFNSYVRVTASDPLDTLGSEGALSPGGVASLLRLPRGCGEATMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDPCPVLDRSMQGGLVGNDETVALTAFVTIALHHGLAVFQDEGAEPLKQRVEASISKANSFLGEKASAGLLGAHAAAITAYALTLTKAPVDLLGVAHNNLMAMAQETGDNLYWGSVTGSQSNAVSPTPAPRNPSDPMPQAPALWIETTAYALLHLLLHEGKAEMADQASAWLTRQGSFQGGFRSTQDTVIALDALSAYWIASHTTEERGLNVTLSSTGR

SEQ ID NO: 9 - protein, 373 aa, Artificial Sequence
  source: /note=“Description of Artificial Sequence Synthetic polypeptide”
MTVARPSVPAALPLLGELPRLLLLVLLCLPAVWGLDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVAWLILEKQKPAGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLSGTTSGTTRLLSGHTCFTLTGLLGTLVTMGLLT

SEQ ID NO: 10 - protein, 373 aa, Artificial Sequence
  source: /note=“Description of Artificial Sequence Synthetic polypeptide”
MTVARPSVPAALPLLGELPRLLLLVLLCLPAVWGLDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKLDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLSGTTSGTTRLLSGHTCFTLTGLLGTLVTMGLLT

SEQ ID NO: 11 - protein, 380 aa, Artificial Sequence; identical to SEQ ID NO: 8
  source: /note=“Description of Artificial Sequence Synthetic polypeptide”

SEQ ID NO: 12 - protein, 368 aa, Artificial Sequence
  source: /note=“Description of Artificial Sequence Synthetic polypeptide”
MTVARPSVPAALPLLGELPRLLLLVLLCLPAVWGLDAERLKHLIVTPSGCGEANMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLENGGTSLSEKTVLLLVTPFLAAAWSLHP

SEQ ID NO: 13 - protein, 763 aa, Artificial Sequence
  source: /note=“Description of Artificial Sequence Synthetic polypeptide”
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPGSGSEFPESWLWNVEDLKEPPKNGISTKLMNIFLKDSITTWEILAVSMSDKKGICVADPFEVTVMGPGQQMTLKIEGDHGARVVLVAVDKGVFVLNKKNKLTQSKIWDVVEKADIGCTPGSGKDYAGVFSDAGLTFTSSSGQQTAQRAELQCPQPSGTTSGTTRLLSGHTCFTLTGLLGTLVTMGLLT

SEQ ID NO: 14 - protein, 698 aa, Artificial Sequence
  source: /note=“Description of Artificial Sequence Synthetic polypeptide”
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPSGTTSGTTRLLSGHTCFTLTGLLGTLVTMGLLT

SEQ ID NO: 15 - protein, 447 aa, Artificial Sequence
  source: /note=“Description of Artificial Sequence Synthetic polypeptide”
MTVARPSVPAALPLLGELPRLLLLVLLCLPAVWGTLEIPGNSDPNMIPDGDFNSYVRVTASDPLDTLGSEGALSPGGVASLLRLPRGCGEATMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDPCPVLDRSMQGGLVGNDETVALTAFVTIALHHGLAVFQDEGAEPLKQRVEASISKANSFLGEKASAGLLGAHAAAITAYALTLTKAPVDLLGVAHNNLMAMAQETGDNLYWGSVTGSQSNAVSPTPAPRNPSDPMPQAPALWIETTAYALLHLLLHEGKAEMADQASAWLTRQGSFQGGFRSTQDTVIALDALSAYWIASHTTEERGLNVTLSSTGRSGTTSGTTRLLSGHTCFTLTGLLGTLVTMGLLT